(19) United States
(12) Patent Application Publication
(10) Pub. No.: US 2008/0287874 A1
(43) Pub. Date: Nov. 20, 2008

(54) CONTROLLING DEAD VOLUME OF A PISTON PUMP USING AN ADJUSTMENT SCREW

(75) Inventor: Ahmed Elmouelhi, Minneapolis, MN (US)

Correspondence Address: MEDTRONIC, INC., 710 MEDTRONIC PARKWAY NE, MINNEAPOLIS, MN, 55432-9924, US

(73) Assignee: Medtronic, Inc., Minneapolis, MN (US)

(21) Appl. No.: 11/750,798

(22) Filed: May 18, 2007

(51) Int. Cl. A61M 37/00

(52) U.S. Cl. 604/154; 29/888.02; 604/246

(57) ABSTRACT

The present invention relates generally to infusion devices. More particularly, the present invention is directed toward an inexpensive design to adjust the dead space of a piston pump by adjusting the end position of the piston's forward stroke. The piston pump may include an actuator member with a piston that articulates between a retracted and a forward position in a piston channel. Movement between the retracted and forward position, a pumping stroke, may expel a known quantity of fluid. Because of manufacturing tolerances, the forward position may not exactly line up with the end of the piston channel, resulting in dead space, or ullage, that may result in less than desired accuracy for fluid volume pumped. Utilization of an adjustable stop or stop that contacts the actuator member at the desired forward position may allow for selective elimination of the ullage and precise adjustment of the fluid pumped.

### FIELD

This invention relates generally to infusion devices. More particularly, the present invention is directed toward an adjustment mechanism to adjust the dead space of a piston pump by adjusting the end position of the piston's forward stroke.

### BACKGROUND

Infusion devices may be used to deliver an infusion media (for example, a medication such as insulin) to a patient. Such devices may be implanted into a patient's body to deliver predetermined dosages of the infusion media to a particular location within the patient's body, for example, in the venous system, the spinal column, or within the peritoneal cavity.

A known infusion device of the type described above includes a drive mechanism that includes a reciprocating pumping element made of a ferrous material. The reciprocating pumping element includes an actuator member with a piston portion that is coupled to an armature portion, also known as a piston actuator or pole. The piston portion is configured to reciprocate within a piston channel when a solenoid coil is alternately energized and de-energized. That is, when the solenoid is energized, magnetic flux causes the actuator to move very quickly (on the order of 2-3 milliseconds) until it reaches a stop member. This corresponds to the pump's forward stroke and results in the delivery of a predetermined dosage of infusion media from an outlet chamber to the patient. When the solenoid is de-energized, the lack of magnetic flux allows the actuator to return to its original position under the force of a spring or other return mechanism. This, in turn, causes the pressure in the piston chamber to fall. The reduced pressure in the piston chamber causes infusion media to flow from a reservoir through an annulus between the actuator piston and the piston cylinder wall to refill the piston chamber, thus equalizing the pressure between the reservoir and the piston chamber and preparing the pump for its next pumping or delivery stroke. This is referred to as the refill stroke. The annulus between the actuator piston and the piston cylinder may be very small (i.e. in the order of 150 to 250 microinches radially), resulting in an outlet chamber refill process that takes between about 1 to 2 seconds. In contrast, the pump's forward (delivery) stroke may be approximately 500 times faster than the refill process.

Manufacturing tolerances in the production of the pump components may result in unwanted ullage (also known as “dead volume” or “dead space”) in the pumping chamber. Ullage may include space that the pump does not physically displace during the forward stroke, resulting in an inaccurate pumped volume. The dead space may lead to trapped air bubbles that are not displaced during the pumping strokes. The air bubbles can further lead to pump dysfunction that is, in part, due to the fact that the trapped air bubbles are compressible. A compressible air bubble in the pumping chamber may result in a smaller amount of displaced fluid from each pumping stroke due to piston movement, resulting in air compression rather than fluid displacement.

A need therefore exists for a method and apparatus to account for manufacturing tolerances in the assembly of the pump components. A need also exists to reduce or eliminate ullage in a piston type pump.

### BRIEF SUMMARY OF THE INVENTION

The present invention includes an apparatus and method for reducing or eliminating ullage in a piston type pump. The apparatus includes an adjustable stop member to selectively adjust the position of the piston during the forward stroke so as to reduce the volume that is not displaced.

On embodiment may be an apparatus for delivering a fluid, the apparatus including a housing, an inlet in the housing for receiving the fluid, an outlet in the housing for discharging the fluid, an actuator positioned within the housing and moveable between a first position and a second position to displace the fluid from the inlet and through the outlet, and an adjustable stop operatively positioned relative to the actuator whereby the second position is selected by adjustment of the adjustable stop.

Another embodiment may include an adjustable actuator for delivering fluid through a piston channel from an inlet to an outlet, the actuator including an armature configured to move between a forward position and a retracted position, a piston coupled to the armature and moveable within the piston channel, and an adjustable stop operably positioned to contact the armature to selectively adjust the forward position.

Yet another embodiment may be a method for manufacturing a piston pump that providing a pump assembly including a housing, an inlet in the housing for receiving the fluid, an outlet in the housing for discharging the fluid, a piston channel within the housing through which the fluid flows from the inlet to the outlet, and an actuator positioned within the housing and moveable between a first position and a second position, the actuator driving the fluid stored in the piston chamber toward the outlet when the actuator transitions from the first position to the second position, the actuator comprising an armature, and a piston coupled to the armature and moveable within the piston channel, placing the actuator in the first position, and adjusting a stop to selectively adjust the position of the actuator in the first position to a desired position in the piston channel.

### BRIEF DESCRIPTION OF THE DRAWINGS

Embodiments of the present invention will hereinafter be described in conjunction with the following drawings wherein like reference numerals denote like elements throughout.

FIG. 1 is an isometric view of an implantable infusion device in accordance with one embodiment of the present invention.

FIG. 2 is a representative view of an infusion device implanted into a body of a patient in accordance with one embodiment of the present invention

FIG. 3 is a cross-sectional view of a drive mechanism in accordance with a first embodiment of the present invention.

FIG. 4 is an exploded view of a portion of the drive mechanism shown in FIG. 3.

FIG. 5 is simplified schematic view of one embodiment of the present invention adjustment mechanism with the actuator of the drive mechanism in a neutral position.

FIG. 6 is simplified schematic view of the drive mechanism of FIG. 5 with the actuator in a forward position.

FIG. 7 is simplified schematic view of the drive mechanism of FIG. 5 with the actuator in a retracted position.

FIG. 8 is simplified schematic view of an alternative embodiment adjustment mechanism of the present invention.

### DETAILED DESCRIPTION OF THE INVENTION

The present invention is an adjustment mechanism for selectively adjusting the forward position of an actuator member of a piston type pump in order to eliminate undesired dead space not pumped during the pumping stroke, also known as ullage. In particular, the adjustment mechanism is an adjustable stop member that can be positioned to contact a portion of the actuator member to halt the actuator in a desired position during a forward pumping stroke. Adjusting the position of the piston during the forward stroke of the actuator member allows for a user to eliminate any unwanted ullage, or dead space that may occur if the piston does not complete its stroke in the desired position.

The following detailed description is of the presently contemplated mode of implementing the invention. This description is not to be taken in a limiting sense, but is merely for the purpose of illustrating the general principles of embodiments of the invention. Furthermore, there is no intention to be bound by any theory presented in the preceding background of the invention or the following detailed description of the invention. The scope of the invention is defined by the appended claims.

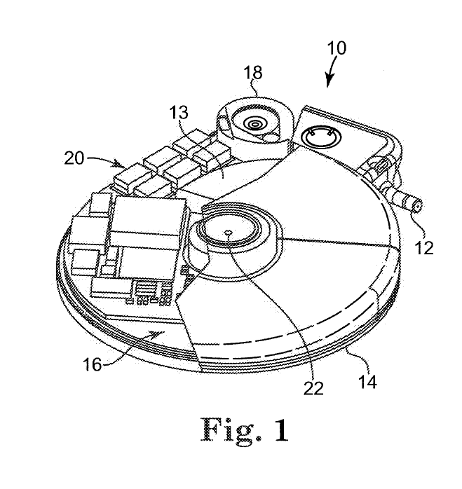

FIG. 1 shows an implantable infusion device 10. The illustrated device 10 is configured to be surgically implanted into a patient, for example, in the abdominal region, between the skin and the abdominal wall. A catheter (not shown) connected to the infusion device 10 deliver infusion medium to the patient, for example, but not limited to, by feeding infusion medium to a particular location in the venous system, within the spinal column, or in the peritoneal cavity of the patient. Other embodiments of the infusion device 10 may be implemented as external infusion devices that connect to patients through suitable catheter devices or the like. Yet further embodiments of the infusion device 10 may be used in other contexts, e.g., for delivery of a medium into other suitable environments. Therefore, for purposes of simplifying the present disclosure, the term “patient” is used herein to refer to any environment in which an implantable device is implanted or to which an external device is connected, whether or not the implant or connection is carried out for medical purposes. Also, the term “infusion medium” is used herein to refer to any suitable medium delivered by the drive device.

In further embodiments, the present invention actuator member may be included in pumping systems not related to infusion devices. An implantable infusion pump, however, will be utilized in the remainder of this description for the sake of simplicity.

A description of the implantable infusion pump and how it is installed in the body may help to provide some further context for the present invention. The device 10 may include a generally disc-shaped housing 14. While a generally circular disc-shaped embodiment is illustrated in FIG. 1, it will be understood that further embodiments of the infusion device 10 may employ housing of other shapes, including, but not limited to, oval, oblong, rectangular, or other curved or polygonal shapes. Generally, the housing 14 is made of a biocompatible material and most often has a relatively small diameter and thickness to reduce patient trauma during implant surgery and after implantation.

The housing 14 includes a reservoir 16 for holding a volume of infusion medium, such as, but not limited to, a liquid medication to be administered to the patient. Housing 14 may also contain a drive mechanism 18 (e.g. a pump), a power source 13, and control electronics 20. Pump 18 may be configured to receive infusion media from reservoir 16 via a pump inlet 22. Inlet structure 22 may provide a closeable and sealable fluid flow path to the reservoir 16 in the reservoir portion of the housing. The inlet structure may include a port for receiving a needle through which fluid may be transferred to the infusion device, for example, to fill or re-fill the reservoir 16 of the device with the infusion media or a rinsing fluid as will be more fully discussed below. In particular embodiments, the inlet structure may be configured to re-seal after a fill or re-fill operation, and to allow multiple re-fill and re-seal operations. One example of an inlet structure is described in U.S. Pat. No. 6,652,510, titled “Infusion Device and Reservoir for Same,” which is incorporated by reference herein in its entirety and for everything it teaches and discloses. However, further embodiments may employ other suitable inlet structures, including, but not limited to, those described in U.S. Pat. Nos. 5,514,103 and 5,176,644, each to Srisathapat et al.; U.S. Pat. No. 5,167,633 to Mann et al.; U.S. Pat. No. 4,697,622 to Swift; and U.S. Pat. No. 4,573,994 to Fischell et al., also incorporated by reference. Representative examples of reservoir housing portions and reservoirs which may be employed in embodiments of the invention are described in the above referred to U.S. Pat. No. 6,652,510, and further embodiments may employ other suitable reservoir configurations, including, but not limited to, those described in the above referred to U.S. Pat. Nos. 5,514,103; 5,176,644; 5,167,633; 4,697,622; and 4,573,994.

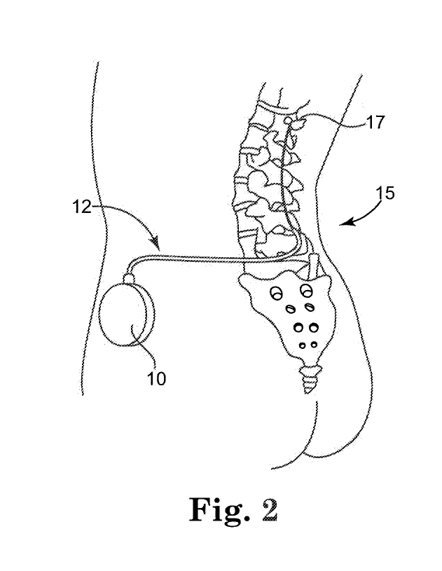

FIG. 2 may illustrate an example placement of one embodiment of an implantable infusion system that is implanted within a patient's body 15. The exemplary infusion systems depicted in implantable medical device 10, and preferably at least one catheter 12. Such infusion systems may be used for a wide variety of therapies including treatment of pain, spasticity, and other medical conditions. Although exemplary infusion systems that may be used in connection with the present invention are described herein, reference may also be had to U.S. Patent Application Publication No. US 2005/0075624 A1, titled “Pressure Sensing in Implantable Medical Devices” (Miesel), which describes infusion systems that may be modified for use accordance with the methods of the present invention.

The medical device 10 and catheter 12 are typically implanted by a clinician (e.g., surgeon) within the body 15 during a surgical procedure. While the present invention also contemplates embodiments wherein the catheter is implanted with a proximal end outside the body 15 so that it may attach to an external infusion device, the remainder of this description is, for the sake of brevity, directed to implantable infusion systems that are entirely implanted in the body 15 of the patient.

Before implantation of the medical device 10, the catheter 12 may be positioned such that the fluid delivered to the patient through the catheter 12 reaches a selected internal delivery location 17 within the body 15 of the patient. As depicted, the infusion system is implanted such that the delivery site 17 is located within the intrathecal space of the spinal canal. As may be appreciated, the infusion systems of the present invention may be used to deliver fluid to any other selected internal delivery location, e.g., epidural, etc.

Catheter 12 may preferably disgorge fluid at other than at its distal end. For example, catheter 12 may intentionally have a delivery region that is not proximate the distal end of the catheter 12, e.g., a hole or valve positioned somewhere before reaching the distal end of the catheter 12. Thus, catheter 12 may be placed in patient with a delivery region of catheter 12 placed in or near to, generally proximate to, the selected internal delivery site 17.

A proximal end of the catheter 12 may be tunneled through the tissue to the device implant location and coupled to a catheter port of the medical device 10. If implanted, the medical device 10 is typically positioned subcutaneously, e.g., from 1 centimeter (0.4 inches) to 2.5 centimeters (1 inch) beneath the skin, where there is sufficient tissue for supporting the medical device 10, e.g., with sutures or the like.

The medical device 10 is, in the illustrated embodiment, operable to infuse a fluid from an enclosed reservoir 16 into the body 15 through the catheter 12.

In order to fully understand the operation of the present invention, a review of an example embodiment pump in which the invention may be utilized may be first helpful.

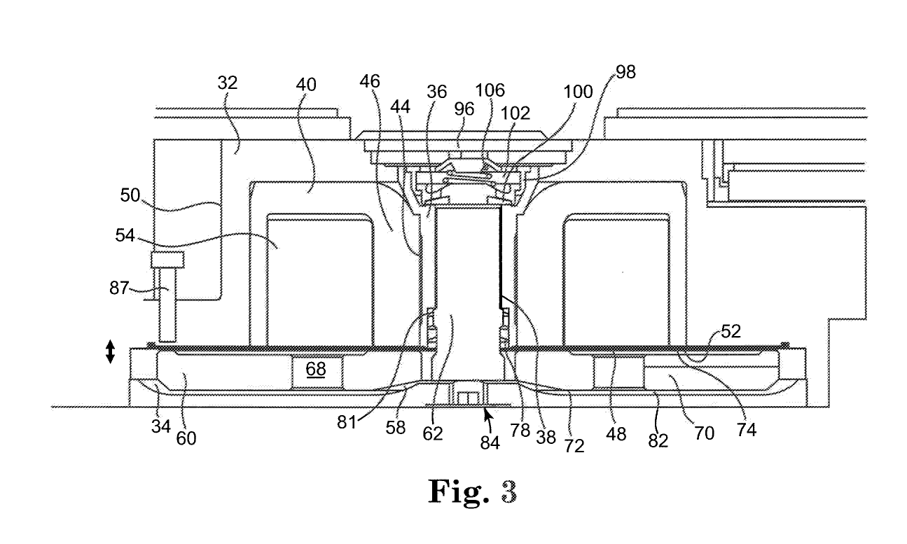

As illustrated in FIG. 3, pump 18 may include an outlet (not shown) through which the infusion medium may be expelled. When the device 10 is implanted in a patient or connected externally to a patient, the catheter 12 may be connected to the outlet to deliver expelled infusion medium into the patient's blood stream or to a selected location in the patient's body. The drive mechanism may be controlled to deliver infusion medium in any suitable manner, for example, according to a programmed dispensing rate or schedule or according to an actuation signal from a sensor, timer or other suitable source.

In particular embodiments, both the drive mechanism 18 and the reservoir 16 may be hermetically sealed. In such embodiments, the housing 14 containing drive mechanism 18 and control electronics 20 may be made from titanium or titanium alloy or other biocompatible metals. The reservoir portion 16 of the housing may be made from similar metals or a biocompatible and infusion medium compatible plastic that allows for the desired hermeticity.

The drive mechanism 18 may include mechanical and electromagnetic components that inhabit a volume of space within the housing 14 in which the components reside and operate. The device 10 is configured such that, once implanted, it functions for a relatively long period of time to administer infusion medium to the patient to periodically be replenished from the outside of patient's body.

As used herein, the term “therapeutic substance” refers to a substance intended to have a therapeutic effect on the patient, e.g., pharmaceutical compositions, genetic materials, biologics, and other substances. “Pharmaceutical compositions,” as used herein, may include chemical formulations intended to have a therapeutic effect such as intrathecal antispasmodics, pain medications, chemotherapeutic agents, and the like. Pharmaceutical compositions are often configured to function effectively in an implanted environment by possessing various characteristics including: stability at body temperature to retain therapeutic qualities; concentration to reduce the frequency of replenishment; and the like. “Genetic materials,” as used herein, may include substances intended to have a direct or indirect genetic therapeutic effect such as genetic vectors, genetic regulator elements, genetic structural elements, DNA, and the like. “Biologics,” as used herein, may include substances that are living matter, or derived from living matter, and offer a therapeutic effect to the patient such as stem cells, platelets, hormones, biologically produced chemicals, and the like. “Other substances” may include most any other substance that is intended to have a therapeutic effect, yet does not clearly fit within one of the categories identified above. Examples of other substances may include saline solutions, fluoroscopy agents, and the like.

In some embodiments, the fluid contained within a reservoir 16 of the medical device 10 may be replenished periodically after device implantation. Typically, replenishment is accomplished with a non-coring needle (not shown) connected to a syringe filled with the fluid. The needle may be inserted through the patient's skin and into a self-sealing septum located within the housing of the medical device 10.

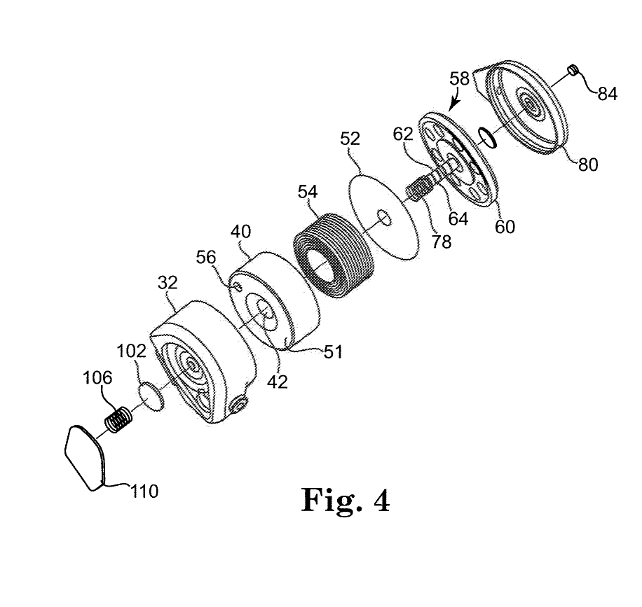

FIG. 3 is a cross-sectional view of a drive mechanism 18 in a retracted position or state. The drive mechanism 18 may employ electromagnetic and mechanical forces to change (or move) between retracted and forward positions or states, also known as first and second positions or states, to cause infusion medium to be drawn in through an inlet and forced out of the outlet 24, respectively. The assembly of components shown in FIG. 3 is also shown in an exploded view in FIG. 4.

Referring to FIGS. 3 and 4, the drive mechanism 18 may include a housing member 32 that is open on one side to a hollow, annular interior section 34. The housing 32 has a central hub portion 36 with a central piston channel 38. The bottom side of the housing member 32 (with reference to the orientation shown in FIG. 3) includes an opening to the hollow interior section 34 through which coil wires may pass. The bottom side of the housing member may also include a configuration of recesses and cavities for providing an outlet chamber and an outlet passage. The housing member 32 is most often made of generally rigid, biocompatible and infusion medium compatible material having no or low magnetic permeability such as, but not limited to, titanium, stainless steel, bio-compatible plastic, ceramic, glass or the like.

As shown in FIGS. 3 and 4, a coil cup 40 is located within the annular interior section 34 of the housing 32. The coil cup 40 may have a generally cylinder shape, open on one side to a hollow, annular interior. The coil cup 40 may include a bore 42 located in a central hub portion 44 and extending axially relative to the annular interior. The hub portion 44 of the cup member defines an inner annular wall 46 having an end surface 48 (or inner pole surface) having a defined width. The cup member 40 has an outer wall 50 having an end surface 52 (or outer pole surface) having a width. The outer wall 50 is connected to the inner wall 46 of hub portion 44 by a backiron portion 51 of the cup member 40. At the open end of cup member 40 the end surfaces 48 and 52 of the inner and outer walls 46 and 50, respectively, define pole surfaces that cooperate with pole surfaces on an armature to provide a path for electromagnetic flux during a forward stroke of the drive mechanism.

When assembled, the coil cup 40 may be located in the hollow interior of the housing member 32, with the central portion 36 of the housing 32 extending through channel 42 of the coil cup 40 as shown in FIG. 3. A coil 54 may be located within the hollow, annular interior of the coil cup 40 and disposed around the axis of the annular interior of the coil cup 40. The coil cup 40 is provided with an opening 56 through which coil leads extend, as shown in FIGS. 3 and 4. The coil cup 40 may be made of generally rigid material having a relatively high magnetic permeability such as, but not limited to, low carbon steel, iron, nickel, ferritic stainless steel, ferrite, other ferrous materials, or the like. The coil 54 may include a conductive wire wound in a coil configuration. The coil wire may include any suitable conductive material such as, but not limited to, silver, copper, gold or the like, with each turn electrically insulated from adjacent turns and the housing. In one particular embodiment, the coil wire may have a square or rectangular cross-section to achieve minimal space between windings and a greater number of coil turns thus improving electrical efficiency.

The drive mechanism 18 may also includes an actuator member 58, which may include an armature portion 60 and a piston portion 62. The actuator member 58 is most often made of a generally rigid, biocompatible and infusion medium compatible material having a relatively high magnetic permeability such as, but not limited to, ferrous materials, ferritic stainless steel with high corrosion resistance, or the like. In the embodiment of FIGS. 3 and 4, the actuator (with an armature portion 60 and a piston portion 62) may be formed as a single, unitary structure.

The armature 60 cooperates with the inner and outer walls of the coil cup 40 to provide a flux path for electromagnetic flux. The spacing between the pole surfaces on the armature 60 and the pole surfaces on the coil cup walls define gaps in the flux path. In particular embodiments, the spacing between the surface of outer pole 70 of the armature 60 and the surface of outer pole 52 of the outer wall 50 of the coil cup 40 is greater than the spacing between the surface of inner pole 72 of the armature and the pole surface 48 of the inner wall 46 of the coil cup (or the barrier 74) when the actuator is in the retracted position shown in FIG. 3.

The radial struts 68 in the armature provide radial paths for electromagnetic flux between the outer and inner pole sections 70 and 72 of the armature. The configuration of openings is most often designed to provide a sufficient conductor for electromagnetic flux and yet minimize or reduce viscous resistance to actuator motion. With reference to FIG. 3, the actuator member 58 is arranged with the piston portion 62 extending through the axial channel 38 of the housing 32 and with the armature portion 60 positioned adjacent to the open side of the coil cup 40. An actuator spring 78 may be positioned to force the armature portion 60 of the actuator 58 in the direction away from the open side of the coil cup 40 to provide a gap between the armature 60 and the open side of the coil cup 40. A biocompatible and infusion medium compatible barrier 74 is located over the open side of the coil cup 40 between the armature 60 and the coil cup 40 to help seal the annular interior of the coil cup 40 and coil 54. In other embodiments in which infusion medium may contact the coil, the barrier 74 may be omitted.

The actuator spring 78 in the illustrated embodiment is a coil spring disposed around the piston portion 62 of the actuator 58 adjacent the armature portion 60. One end of the coil spring abuts the armature portion 60 of the actuator, while the opposite end of the coil spring abuts a shoulder 81 in the piston channel 38 of the housing 32. In this manner, the actuator spring 78 imparts a spring force between the housing and the actuator 58 to urge the actuator toward its retracted position shown in FIG. 3.

In the illustrated embodiment, by using a coil spring 78 located around and coaxial with the piston portion 62 and disposed partially within the piston channel 38, the actuator spring may have minimal or no contribution to the overall thickness dimension of the drive mechanism. However, in other embodiments, actuator springs may have other suitable forms and may be located in other positions suitable for urging the actuator toward its retracted position shown in FIG. 3. The actuator spring 78 is most often made of a biocompatible and infusion medium compatible material that exhibits a suitable spring force such as, but not limited to, titanium, stainless steel, MP35N cobalt steel or the like.

The drive mechanism 18 may further include a cover member 80 which attaches to the housing member 32 over the open side of the housing member and the barrier 74. The cover member 80 is most often made of a generally rigid, biocompatible and infusion medium compatible material having a relatively low magnetic permeability (being relatively magnetically opaque) such as, but not limited to, titanium, stainless steel, biocompatible plastic, ceramic, glass or the like.

The cover member 80 defines an interior volume 82 between the barrier 74 and the inner surface of the cover member. The armature portion 60 of the actuator member 58 resides within the interior volume 82 when the cover is attached to the housing. The armature 60 is moveable in the axial direction within the volume 82 between the retracted position shown in FIG. 3 and the forward stroke position, which is described in more detail below. This movement is created by the action of electromagnetic force generated when a current is passed through the coil 54 and the mechanical return action of the actuator spring 78.

A first adjusting stop 84, or adjustable plunger, may be located within the cover 80 for contacting the armature 60 to set the retracted position of the armature when the armature is in the fully retracted position shown in FIG. 3. In particular embodiments, a seal (e.g. a silicon rubber sealing ring) may be disposed between the first stop 84 and the cover member 80. In further embodiments, a flexible diaphragm (not shown) (such as, but not limited to, a thin titanium sheet or foil) may be coupled to the inside surface of the cover 80 and sealed around the opening through which the first stop 84 extends. The diaphragm will flex to allow the stop to define an adjustable retracted position while also providing sealing functions for inhibiting leakage at the interface between the first stop 84 and the cover 80. In other embodiments, after a proper armature position is set, the stop is fixed in place with respect to the cover member, for example, by adhering the stop to the cover member with one or more welds, adhesives or other securing methods.

As is further illustrated, in the present invention the pump 18 includes a second adjusting stop 87. The second stop 87, also known as an adjustable plunger, adjustable ullage stop, or ullage control, may be positioned to selectively adjust the position of the actuator member 58 at the desired forward stroke position during pumping. As is further described below, adjustment of the second stop 87 may reduce the ullage present in the infusion device 10 and therefore result in a more accurate pumping device. As may be appreciated, the second stop 87 may be located at other positions that allow for contact with the actuator member 58 to perform the adjustment.

As shown in FIG. 3, the piston portion 62 of the actuator 58 may extend through the axial channel 38 in the housing 32 toward an outlet at the end of the axial channel 38. The channel 38 may have an inside diameter which is larger than the outside diameter of the piston portion 62. As a result, an annular volume is defined between the piston portion 62 and the wall of the axial channel 38 along the length of the axial channel 38. Infusion medium may flow through the annular volume 82 within the cover 80 to a piston chamber 100 located between the free end of the piston portion 62 and a valve member 102 of a valve assembly 96. In particular embodiments, the radial spacing between the piston portion 62 and the wall of the channel 38 is selected to provide a suitable flow toward the piston chamber 100 to refill the piston chamber 100 (during a return stroke of the piston portion), but small enough to sufficiently inhibit back flow of medium from the piston chamber 100 (during a forward stroke of the piston portion).

The actual radial spacing between the piston portion 62 and the wall of the channel 38 to achieve such results depends, in part, on the overall dimensions of those components, the pressure differentials created in the mechanism, and the viscosity of the infusion medium.

The valve assembly 96 in the embodiment of FIG. 3 may further include the valve member 102 and a valve spring 106. The valve member 102 is located within the outlet chamber 98 and, as shown in FIG. 3, is positioned to close the opening between the axial channel 38 and the outlet chamber 98 when the actuator 58 is in the retracted position. During the forward stroke, the valve member 102 is positioned to open a flow passage between the axial channel 38 and the outlet chamber 98. The valve spring 106 is located within the outlet chamber 98 to support the valve member 102. The spring 106 imparts a spring force on the valve member 102 in the direction toward piston 62 urging the valve member 102 toward a closed position to block the opening between the axial channel 38 and the outlet chamber 98.

The valve member 102 is most often made of generally rigid, biocompatible and infusion medium compatible material, such as, but not limited to, titanium, stainless steel, biocompatible plastic, ceramic, glass, gold, platinum or the like. A layer of silicon rubber or other suitable material may be attached to the rigid valve member material on the surface facing the channel 38 to help seal the opening to channel 38 when the valve member is in the closed position shown in FIG. 3.

The valve spring 106 is most often made of biocompatible and infusion medium compatible material that exhibits a suitable spring force such as, but not limited to, titanium, stainless steel, MP35N cobalt steel or the like. In the illustrated embodiment, the valve spring 106 is a coil spring. In other embodiments, other suitable valve spring configurations may be employed, including, but not limited to, helical, flat, radial, spiral, barrel, hourglass, constant or variable pitch springs or the like.

The embodiment shown in FIG. 3 utilizes a valve cover 110 sealed to the housing 32 to enclose the outlet chamber 98. The valve cover 110 is most often made of a generally rigid, biocompatible and infusion medium compatible material, such as, but not limited to, titanium, stainless steel, biocompatible plastic, ceramic, glass, gold, platinum or the like.

The coil 54 may be inserted into the annular interior of the coil cup 40 with the coil leads extended through a coil lead opening 56 in the coil cup. The coil may be impregnated or partially impregnated with a fill material of epoxy or the like for adhering the coil to the coil cup and for sealing or partially sealing the coil. The fill material may also be used to adhere the barrier plate to the coil members to avoid warping or bulging of the barrier plate after assembly.

The coil cup 40 and the coil 54 may be inserted into the interior of the housing 32 with the coil leads (which may be wire leads or flexible conductive tabs) extending through a coil lead opening 56 in the housing 32. In particular embodiments, the coil cup and housing are configured to provide a tight friction fit that does not require additional means to adhere the two components together. In other embodiments, the coil cup 40 and housing 32 may be coupled together by a suitable adhesive material or other adhering methods, including, but not limited to, welding, brazing or the like.

The barrier 74 may be placed over the coil, coil cup and housing sub-assembly. The barrier 74 may be adhered to the housing by one or more adhering points or continuously secured along the circumference of the barrier 74 with any suitable adhesive material or other adhering methods including, but not limited to, welding, brazing, soldering, or the like. Alternatively, or in addition, the barrier 74 may be held in place by a shoulder portion of the cover 80, as shown in FIG. 3. In addition, as noted above, the barrier 74 may be adhered to the coil 54 by fill material in the coil. In particular embodiments, the barrier 74 is held in a generally flat position relative to the coil cup and coil. To enhance this flat relationship, the coil cup and housing may be assembled together and then machined to planarize the barrier contact surfaces prior to inserting the coil in the coil cup and prior to adding fill material to the coil.

After the barrier 74 is placed over the coil, coil cup and housing, the actuator 58 may be added to the sub-assembly. First, however, the actuator spring 78 is placed around the piston portion 62 adjacent the armature portion 60 of the actuator. Then the free end of the piston portion 62 is passed through the axial channel 38 of the housing 32 with the armature end of the actuator arranged adjacent the barrier 74.

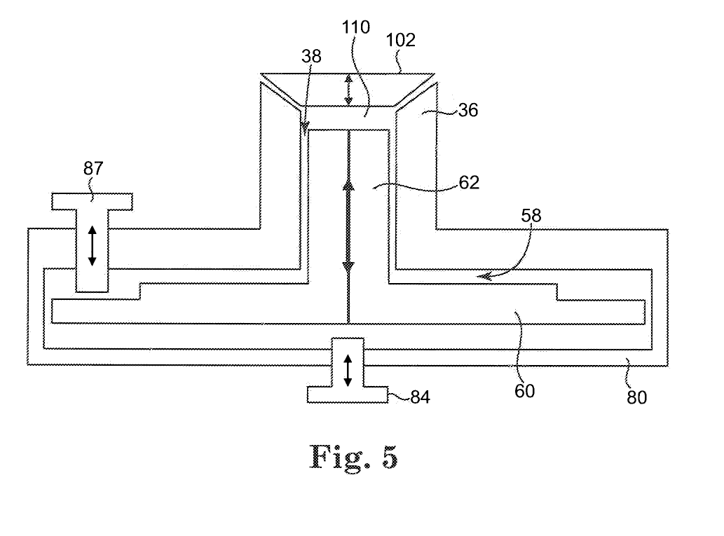

A simplified schematic of the actuator member 58, including the armature portion 60 and the piston portion 62, is illustrated in FIG. 5. Drive mechanism 18 employs electromagnetic and/or mechanical forces to move between a retracted, or first, position, wherein the actuator 58 is in contact with the first stop 84, and a forward, or second, position, wherein the actuator 58 (armature 60) is in contact with the second stop 87, to cause infusion medium to be drawn into and driven out of the pump 18 and the infusion device 10 in a controlled manner.

The position of the actuator 58 in the retracted position may be adjusted by adjusting the position of the first stop 84. In one particular embodiment, adjusting the first stop 84 includes adjusting a threaded cylindrical member that engages corresponding threads in the cover member 80. An exposed end of the first stop 84 may be provided with a tool-engagement depression for allowing engagement by a tool, such as a screw-driver, Allen wrench or the like, from outside of the cover member 80. By engaging and rotating the first stop 84 with a suitable tool, the depth that the stop extends into the cover member 80 may be adjusted to adjust the retracted position of the armature portion 60 and therefore the piston 62 in the piston channel 38. In one particular embodiment, adjustments of the first stop 84 are made during manufacture. In that embodiment, the adjusted position is set by welding or otherwise adhering the first stop 84 in the adjusted position during the manufacture. In other embodiments, the first stop 84 is not set and welded during manufacture to allow adjustment of the first stop 84 after manufacture.

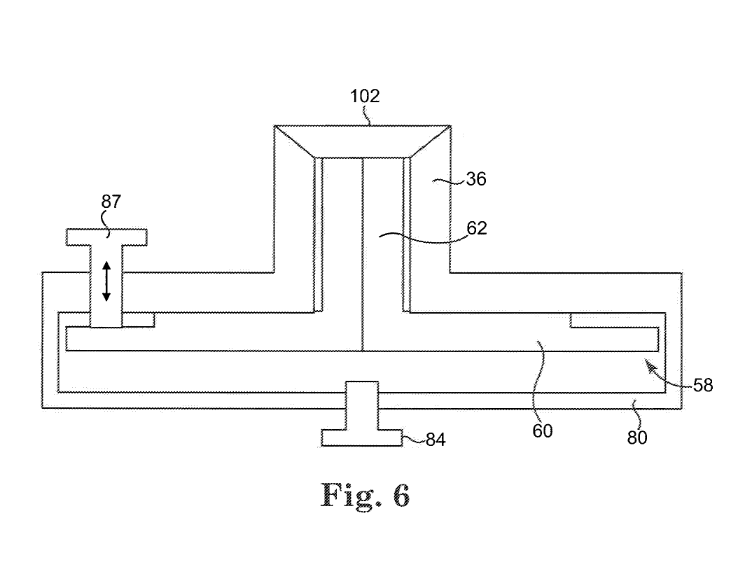

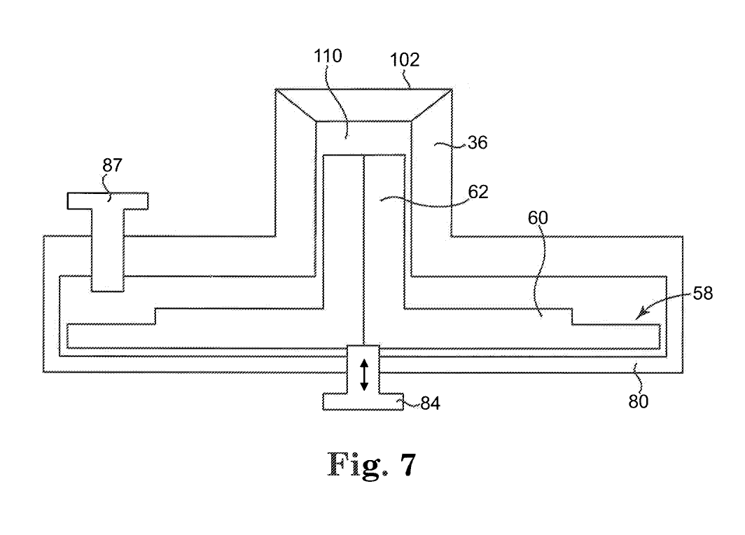

In the present invention, the second stop 87 may be adjusted to help adjustably select the volume pumped during each pumping stroke, and eliminate any unwanted ullage, by adjusting the position of the piston 62 at the end of the forward stroke. The pumped volume is represented by space 110 in FIGS. 6 and 7. As previously described in relation to stop 84, one end of the second stop 87 may be provided with a tool-engagement depression for allowing engagement by a tool in the same manner as the first adjustment 84. By engaging and rotating the second stop 87 with a suitable tool, the position that the stop 87 contacts the armature portion 60 may be adjusted to adjust the forward position of the armature portion 62, the piston 60, and therefore the ullage. The adjustment of the second stop 87 may also be made during manufacture and set by welding or otherwise adhering the second stop 87 in the adjusted position or left adjustable to allow adjustment of the second stop 87 after manufacture.

During manufacturing, the position of the second stop 87 may be adjusted before the position of the first stop 84. In such an embodiment, the second stop 87 may be adjusted to set the forward position of the stroke for the actuator member 58. In the illustrated embodiment, the actuator member 58 forward stroke is limited by the armature portion 60 contacting the second stop 87. This end of forward stroke position may be selected such that the end of the piston 62 when the actuator 58 completes its pumping cycle is in a desired relationship with the end of the piston chamber. For example, as illustrated in FIG. 6, the position can be selected such that the forward, second, position does not leave any gap between the piston 62 and the valve member 102 at the end of the piston channel 38, reducing or eliminating the unwanted ullage. During adjustment, the actuator 58 may be manually positioned in the forward position to adjust the second stop 87 or the pump may be activated whereby the actuator 60 is moved by the coils.

Once the forward position for the actuator 60 is selected, the volume of fluid to be pumped during each pumping stroke, represented by space 110, may then be selected. The volume is selected by moving the actuator 60 towards the first position such that the desired space 110 is left between piston 62 and valve 102. The first stop 84 is then adjusted to contact the actuator 58 at the desired actuator 58 position.

In one embodiment, the piston 62 may be purposefully made a little longer than necessary so as to require the actuator 58 to be dialed back so as to bring the piston 62 into an aligned position with piston channel 38 and the valve member 102.

Because of manufacturing tolerances, adjusting the forward and retracted positions of the actuator 58 in this manner may help to insure that each manufactured pump dispenses the same or nearly the same volume of fluid during each pumping stroke. The total volume of delivered fluids by the pump may therefore be extremely accurate.

The first and second stops 84 and 87 may be accurately adjusted using a variety of methods. One method may comprise the stops 84, 87 as screws such that each turn is equal to a set distance. In other embodiments the adjustment stops 84, 87 may include detents to indicate depths. Other adjustable stops with different kinds of indicators may be substituted without changing the nature and scope of the present invention.

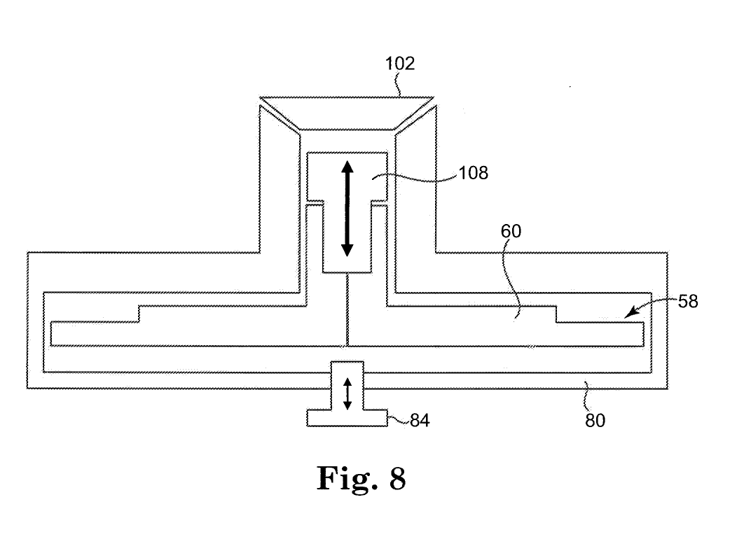

In further embodiments, an adjusting member 108 may be included on the actuator member 58 to help control ullage. In one embodiment illustrated in FIG. 8, the adjustable stop may extend from the piston 62. The adjustable extension 108 may adjust the length of the piston 62 whereby the end of the piston 62 is at the desired location at the end of the forward stroke. Such an adjustable extension 108 may include a variety of different engagement mechanisms, such as a screw thread, sliding engagement means, or others. Such and adjusting member 108 may also be incorporated into pump 18 in conjunction with the second stop 87 may also be loaded on armature 60.

While at least one exemplary embodiment has been presented in the foregoing detailed description of the invention, it should be appreciated that a vast number of variations exist. It should also be appreciated that the exemplary embodiment or exemplary embodiments are only examples and are not intended to limit the scope, applicability, or configuration of the invention in any way. Rather, the foregoing detailed description will provide those skilled in the art with a convenient road map for implementing exemplary embodiments of the invention, it being understood that various changes may be made in the function and arrangement of elements described in an exemplary embodiment without departing from the scope of the invention as set forth in the appended claims and their legal equivalents.

What is claimed is:

1. An apparatus for delivering a fluid, the apparatus comprising:
a housing;
an inlet in the housing for receiving the fluid;
an outlet in the housing for discharging the fluid;
an actuator positioned within the housing and moveable between a first position and a second position to displace the fluid from the inlet and through the outlet; and
an adjustable stop operatively positioned relative to the actuator whereby the second position is selected by adjustment of the adjustable stop.

2. The apparatus of claim 1 wherein the actuator further comprises a piston and an armature.

3. The apparatus of claim 1 wherein the adjustable stop is operatively positioned to contact the armature.

4. The apparatus of claim 1 further comprising a piston channel, the piston moving in the piston channel between a first position and a second position to displace the fluid.

5. The apparatus of claim 1 further comprising a valve member operably positioned at one end of the piston channel.

6. The apparatus of claim 1 wherein the adjustable stop is a screw.

7. The apparatus of claim 1 wherein the adjustable stop further comprises depth indicators.

8. The apparatus of claim 1 wherein the adjustable stop further comprises a tool engagement depression for adjusting the adjustable stop.

9. An adjustable actuator for delivering fluid through a piston channel from an inlet to an outlet, the actuator comprising:
an armature configured to move between a forward position and a retracted position;
a piston coupled to the armature and moveable within the piston channel; and
an adjustable stop operably positioned to contact the armature to selectively adjust the forward position.

10. A method for manufacturing a piston pump comprising:
providing a pump assembly including:
a housing;
an inlet in the housing for receiving the fluid;
an outlet in the housing for discharging the fluid;
a piston channel within the housing through which the fluid flows from the inlet to the outlet; and
an actuator positioned within the housing and moveable between a first position and a second position, the actuator driving the fluid stored in the piston chamber toward the outlet when the actuator transitions from the first position to the second position, the actuator comprising:
an armature; and
a piston coupled to the armature and moveable within the piston channel;
placing the actuator in the first position; and
adjusting a stop to selectively adjust the position of the actuator in the first position to a desired position in the piston channel.
